(19) United States
(12) Patent Application Publication
(10) Pub. No.: US 2026/0248653 A1
(43) Pub. Date: Aug. 27, 2026

(54) EYEWEAR DEVICE FOR BIOPOTENTIAL MEASUREMENT

(71) Applicant: Snap Inc., Santa Monica, CA (US)

(72) Inventors: Sid Kouider, Paris (FR); Guillaume Ployart, Paris (FR); Bastien Rechke, Paris (FR); Adrien Jollet, Paris (FR)

(21) Appl. No.: 17/809,351

(22) Filed: Jun. 28, 2022

Related U.S. Application Data
(60) Provisional application No. 63/215,613, filed on Jun. 28, 2021.

(51) Int. Cl. A61F 9/04; G02C 1/02; G02C 5/12

(52) U.S. Cl. CPC A61F 9/04; G02C 1/02; G02C 5/126; G02C 2200/16

(57) ABSTRACT

Eyewear for capturing and processing electroencephalogram (EEG) signals. The eyewear includes one or more biopotential measurement systems attached to rearward extensions of the temple arms of the eyewear. The biopotential measurement systems include an electrode arrangement that contacts the scalp of a user of the eyewear for capturing EEG signals of the user. The eyewear further includes a reference electrode mounted on a frame of the eyewear and in contact with the nose of the user. The eyewear processes the EEG signals to provide user inputs into software applications.

### CROSS REFERENCE TO RELATED APPLICATION

This application claims the benefit of priority of U.S. Provisional Application Ser. No. 63/215,613, filed on Jun. 28, 2021, and titled “EYEWEAR DEVICE FOR BIOPOTENTIAL MEASUREMENT”, the contents of which are incorporated by reference herein.

### TECHNICAL FIELD

The present application relates to head-mounted devices used in the acquisition of diffuse electrical signals. In particular, biopotential measurement in an electroencephalogram (EEG) acquisition eyewear device.

### BACKGROUND

Surface electroencephalography makes it possible to measure the variations of diffuse electric potentials on the surface of the skull of a subject. These variations of electrical potentials are commonly referred to as electroencephalogram signals or EEG signals.

EEG signals may be used as user inputs into software applications. Accordingly, convenient methodologies for capturing and processing EEG signals are desirable.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an electronic architecture for receiving and processing EEG signals in accordance with some examples of the present disclosure.

FIG. 2 shows a schematic diagram of an acquisition subsystem for capturing and digitizing EEG signals in accordance with some examples of the present disclosure.

FIG. 3 shows a schematic arrangement of an active dry electrode in accordance with some examples of the present disclosure.

FIG. 4A shows an eyewear device in accordance with some examples of the present disclosure.

FIG. 4B shows an eyewear device in accordance with some examples of the present disclosure.

FIG. 5 shows detail of an electrode arrangement for an eyewear device in accordance with some examples of the present disclosure.

FIG. 6 shows detail of a frame of an eyewear device in accordance with some examples of the present disclosure.

FIGS. 7A and 7B show temple adjustment arrangements for eyewear devices in accordance with some examples of the present disclosure.

### DETAILED DESCRIPTION

Surface electroencephalography makes it possible to measure the variations of diffuse electric potentials, such as EEG signals, on the surface of the skull of a subject.

The measured EEG signals may be used to monitor states of brain activity. In some cases, the subject can exercise control over the states of brain activity so that the EEG signals can be used to infer the conscious intention of the subject. In this way, an EEG device may form part of a brain-computer interface (BCI) system. In brain-computer interfaces (BCIs), neural responses to a target stimulus, generally among a plurality of generated stimuli presented to the user, are used to infer (or “decode”) which stimulus is essentially the object of focus at any given time. The object of focus can then be associated with a user-selectable or controllable action.

In one category of BCI systems, the stimulus responsible for the monitored neural response is visual, so that the BCI system incorporates a display device, which may for example be the display of a computing system or smart phone, a television display, or a head-mounted display or eyewear for providing an augmented reality (AR) or virtual reality (VR) experience. Interpreted EEG signals may be used as input signals for a processing device that controls graphical elements displayed in the display or otherwise interacts with the display device.

EEG devices in the medical or related research fields may include a close-fitting acquisition headset with attachment locations for receiving individual sensors/electrodes. Electronic circuits are then connected to the electrodes via cables and to the housing of an acquisition chain comprising an assembly of connected components used in acquiring the EEG signals.

Every time EEG acquisition headsets are worn the placement of the electrodes will vary. In turn, the relative electrical properties of the respective electrodes may vary. For this reason, it is usual to provide an electrode whose signal is designated as a reference. By choosing a reference that is relatively unaffected by the activity being measured but likely to be exposed to the same external electrical conditions, the effects of those external conditions may be excluded or indeed cancelled out.

Many EEG acquisition headsets can be cumbersome to wear for lengthy periods and in some cases unacceptable to the wearer. The physiology and mobility of the wearer may prevent the wearer for using such headsets (as may be the case with those whose heads need to be braced by a headrest to avoid discomfort or injury).

Where the EEG device is to monitor a particular class of brain activity, such as neural signals related to visual stimuli, it is possible to make the EEG device more compact. Visual processing in the brain is primarily performed in the visual cortex close to the back of the brain, so that an EEG device for detecting neural responses to visual stimuli is most effectively deployed at the rear of the head that is a portion of the head closest to the visual cortex. Typically contact between the wearer's head and EEG sensors may be maintained by attaching the sensors to an elasticated head band or helmet worn about the head, thereby biasing the sensors against the head. The band or helmet itself may give rise to discomfort and may be inconvenient in regular use-interfering with other wearable devices, headgear and ornamentation the wearer might already carry or wear (such as hats, jewelry, hairstyles, headphones, face masks, etc.).

Compact EEG devices having a form factor that is commensurate with the portion of the brain they monitor do give rise to a problem of identifying suitable electrode placements to be designated as reference. The compact nature of these devices means that available electrode placement sites are likely to be closer to the area of interest than would be ideal: leading to a reduction in the signal to noise ratio that can be achieved.

It is a further challenge for any headset arrangement to be able to apply a predetermined biasing force to the sensors regardless of the head shape and/or head dimensions of the wearer.

Extracting a reliable EEG signal, given the small amplitude of the electrical potential variations to be measured that may of the order of a few microvolts, drives development of surface EEG equipment that improves conductivity between sensors and the scalp and reliability of the contact under circumstances that vary over time. One obstacle to that improved contact can be condition, such as the length, thickness and style, of the hair of the subject, which can significantly affect the impedance experienced by the measured electrical potentials. Another obstacle can be the physiology and mobility of the subject. Worn over any prolonged period, the contacts can be degraded by perspiration and the subject may be tempted to adjust the headset positioning for comfort.

To address the issue of signal reliability, some surface EEG devices are equipped with gel electrodes, in which contact is made through a gel or conductive liquid, which easily seeps through the user's hair to reach the scalp. The electrode itself is generally made of metal. The gel makes it possible to reduce the electrical impedance and thus the interference with surrounding signals without requiring physical contact between the electrode and the scalp. This solution provides good conductivity at any point of the scalp. Technical assistance is however required to ensure appropriate electrode placement, which in turn is time-consuming (since the gel must be applied and the conductance checked individually for each electrode).

In some examples, the electrode includes circuitry (in addition to the metal “sensor” itself). This circuitry may process the analog signal coming from the sensor to amplify the electrical potentials captured at the sensor before that signal is routed to other electronic components. Such electrodes are termed the “active” wet electrodes. Amplification, in this context, means that a component strongly drives the voltage on a line to the level coming from the sensor. After amplification, the signal is less susceptible to interference as it is conveyed along connector cables to the other components before conversion (from analog to digital). The greater the impedance (due to hair condition, for example), the weaker the signal (in terms of driving voltage on the line) so this amplification circuitry can be essential for proper functioning even with gel.

The use of gel limits the duration of use of the device to a few hours (since the contact is no longer assured as the gel dries). In many cases, the conductive liquid or gel leaves a residue in the hair after the use of the EEG device, this can be difficult to remove.

More recently, surface electroencephalographs equipped with so-called “active dry” electrodes have been introduced: the electrodes being termed “dry” because they require no gel or other conductive liquid. Active dry electrodes operate by capturing the variations of electrical potential signals on the surface of the scalp, and then amplifying those signals (in some cases also filtering the signals). The analog signals thus obtained are then converted into digital signals by means of one or more analog-to-digital converters controlled by a microcontroller. The microcontroller receives the data for analysis, storage and/or onward transmission to another device.

In active dry electrodes, the contact with the scalp is through solid conducting elements or “sensors” connected to an electronic circuit to overcome the increase in impedance (compared to impedance in the presence of gel). The active dry electrode facilitates signal capture comparable to that of a gel electrode but also allows filtering and/or amplification of the captured signals, and thus an improved signal-to-noise ratio.

Stable access to the scalp limits the reliability of the signal capture. The shape of the sensors is restricted by the need for contact that extends through the hair of the subject.

It is known to provide pin-style active dry sensors in a conductive polymer material that require the application of significant pressure to reach the user's scalp. Such sensors are uncomfortable, however, (particularly for prolonged use) because of the application of pressure at the interface of scalp and sensor pin.

Medical and research EEG devices often use acquisition headsets in the form of an unflattering cap, often of elastic or waterproof fabric, with attachment locations for receiving individual sensors/electrodes. The EEG device therefore comprises electrodes (in a headset arrangement together with connecting cables), circuitry for digitizing, amplifying and/or filtering the signals captured at the electrodes and a microcontroller for processing the filtered transmission to an external processing unit. The EEG device is thus typically formed of three distinct elements that an operator/exhibitor must assemble at each use. Again, the nature of the EEG device is such that technical assistance is desirable if not essential.

Furthermore, user acceptability of the EEG device (and its electrodes) places aesthetic constraints, as well as constraints in comfort and ease of use. In many cases, these constraints are an effective significant barrier to the adoption of EEG technology. Examples of applications where comfort over prolonged use and the need for technical assistance prevent adoption include applications such as video games, training (e.g., for health and safety or flight simulation), sleep aids, etc.

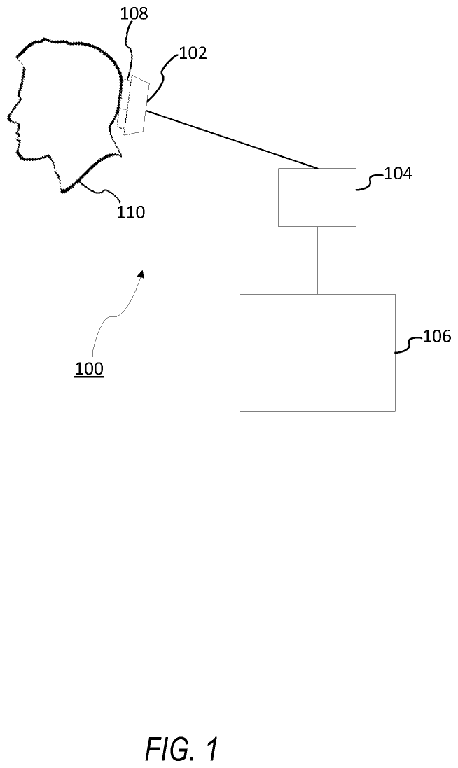

FIG. 1 illustrates an example of an electronic architecture for the reception and processing of EEG signals by means of an EEG device 100.

To measure diffuse electric potentials on the surface of the skull of a subject user 110, the EEG device 100 includes a portable device 102 (e.g., a cap, a headpiece, or the like), analog to digital converter (ADC) circuit 104 and a microcontroller 106. The portable device 102 of FIG. 1 includes one or more active dry electrodes 108, typically between 1 and 128 electrodes, advantageously between 2 and 64, or advantageously between 4 and 16.

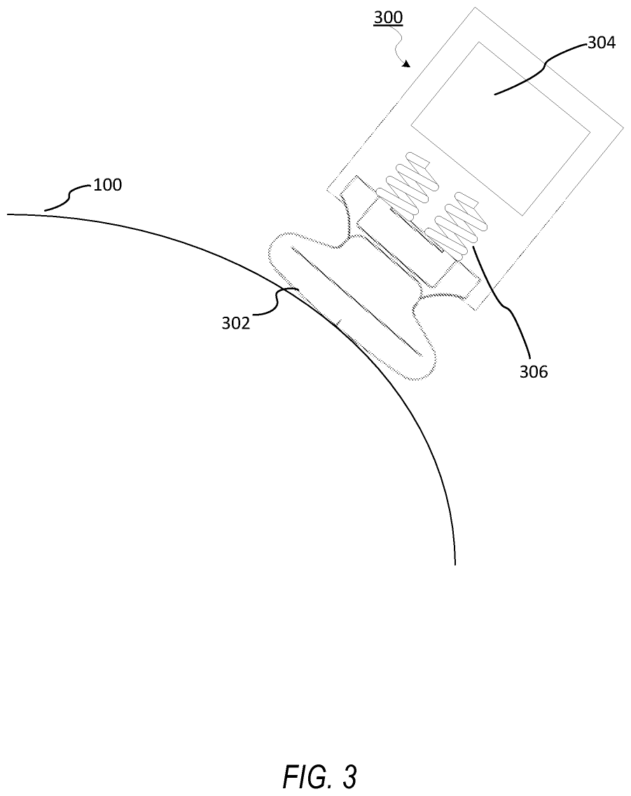

Each active electrode 108 comprises a sensor for detecting the electrical signals generated by the neuronal activity of the subject user 110 and an electronic filtering and amplifying circuit. These elements are discussed in more detail in relation to FIG. 3 below. The active electrodes 108 are shown in use in FIG. 1, where the sensor is in physical proximity with the scalp of the subject user 110.

Each ADC circuit 104 is configured to convert the signals of a given number of active electrodes 108, for example between 1 and 128.

Each ADC circuit 104 is controlled by the microcontroller 106 and communicates with it for example by the protocol SPI (“Serial Peripheral Interface”). The microcontroller 106 packages the received data for transmission to an external processing unit (not shown), for example a computer, a mobile phone, a virtual reality headset, an automotive or aeronautical computer system, for example a car computer or a computer system. Airplane, for example by Bluetooth®, Wi-Fi (“Wireless Fidelity”) or Li-Fi (“Light Fidelity”).

The external processing unit may be provided in a brain-computer interface (BCI) system. In visual brain-computer interfaces (BCIs), neural responses to a target stimulus, generally among a plurality of generated visual stimuli presented to the user, are used to infer (or “decode”) which stimulus is essentially the object of focus at any given time. The object of focus can then be associated with a user-selectable or user-controllable action. The biopotential measurements from a device such as the EEG device 100 of FIG. 1 may be used to measure those neural responses.

In some examples, each active electrode 108 is powered by a battery (not shown in FIG. 1). The battery is conveniently provided in a housing of the portable device 102.

As discussed above, active dry electrodes make contact with the scalp of a subject user 110 through solid conducting elements or “sensors” connected to an electronic circuit. The sensors facilitate signal capture, while the corresponding electronic circuits allow amplification and/or filtering of the captured signals.

One or more of the active dry electrodes 108 are configured as “reference” electrodes (i.e., electrodes that assist in providing a reference level of electrical activity against which measurement may occur). Each reference electrode is connected to the ADC circuit 104. While any one of the electrodes at any location could be used as reference, it is good practice to select the reference with care because any activity in the reference electrode will reflected in the activity at other electrodes. The reference electrode or electrodes are preferably positioned in contact with the user's head in a region remote from that of the other active electrodes. In certain arrangements, the ADC circuit 104 includes a differential amplification module and a digitization module. Each active (i.e., measurement) electrode 108 measures a respective electric potential value from which the potential measured by the reference electrode (Ei=Vi−Vref) is subtracted by the differential amplification module, and this difference value is digitized by means of the digitization module of the ADC circuit 104 then transmitted by the microcontroller 106.

In some examples (as illustrated in FIG. 1), the ADC circuit 104 is provided in a single chip capable of performing both differential amplification and digitization. While not illustrated, the respective processes may be performed by separate components. In differential amplification, the reference potential is subtracted from the measuring electrode potential: in some cases, a gain may also be applied. The output of the differential amplification operation is a voltage value (i.e., a potential value referenced to the system's ground). In another example, during digitization by analog to digital conversion, the ADC circuit 104 samples the differential measurement and translates this voltage value into a digital value.

In certain arrangements of electrodes for EEG devices, it may be convenient to provide more than one dedicated reference electrode. Conveniently, each dedicated reference electrode may take the same form as the measurement electrodes. The signal from any one or all of the reference electrodes may be used as a reference signal against which the potential difference at each of the measurement electrodes is measured.

The surface electrical signals emitted by the brain, as measured by EEG devices, are small, often as small as several micro-volts, so it becomes important to reject common-mode voltages which might otherwise swamp the desired signals. The signals measured by the electrodes of EEG devices are equally sensitive to environmental interference-in particular, mains power at 50-60 Hz-also referred to as common mode interference (CMI). As the body of the subject user 110 is electrically floating, electromagnetic signals can couple to the body of the subject user 110, the cables and the circuitry of the EEG device, causing the potentials to drift relative to a reference voltage in the EEG device, affecting the performance of all electrodes in common. To address CMI, EEG devices often include additional circuitry that drives a current that actively cancels the interference (for instance, by monitoring the changing common voltage and driving a reference electrode at a voltage that mirrors the changing voltage around a known reference voltage). This driven current is applied through a physical electrode located at a point distant from the region of the brain being measured.

As driven reference electrodes are also present in the earlier ECG/EKG (electrocardiogramachnologies and the distant point for such technologies was the right leg (the right leg being the part of the body which is furthest from the heart), the circuitry is termed the “driven right leg circuit” or DRL, even when discussing EEG technologies. Electrodes paired with the reference electrode through this circuit are used as a neutral lead in EEG. Usually, the known reference voltage is half the supply voltage.

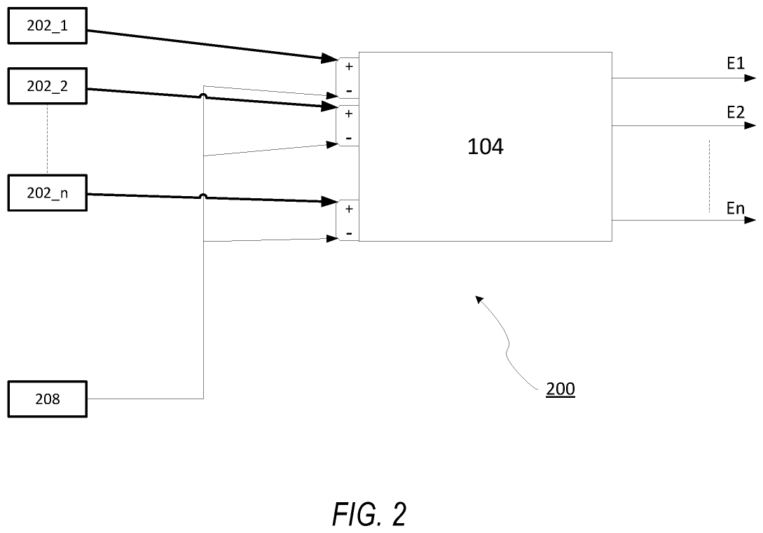

FIG. 2 shows a schematic diagram of an acquisition subsystem 200 for capturing and digitizing EEG signals in an EEG device, such as the one illustrated in FIG. 1. For simplicity, the acquisition subsystem 200 of FIG. 2 uses one specific electrode 208 as reference for all other measuring electrodes 202_1, 202_2, . . . 202_n (collectively measuring electrodes 202). This reference electrode 208 is connected to the negative input pins of all the channels of the ADC circuit 104, where each positive input is connected to a respective measuring electrode 202.

In some examples, the acquisition subsystem 200 changes which electrode is considered as a reference against which all other electrodes potentials are measured. In some examples, where none of the electrodes 202_1 to 202_n can be relied upon as a permanent reference electrode, acquisition subsystem 200 takes an average of activity at all of the electrodes 202_1 to 202_n as a reference.

FIG. 3 illustrates an arrangement of functional elements of an active dry electrode 300 including a sensor 302 and an electronic circuit 304 in accordance with some examples. The active dry electrode may be used as one of the one or more active dry electrodes 108 in FIG. 1 or 202_1 . . . 202_n in FIG. 2.

In some examples, each electronic circuit 304 comprises a first-order high-pass analog filter, an amplifier and a first-order low-pass analog filter. The filters make it possible to suppress signals that are detected by the frequency components that are less useful for the intended application.

The active dry electrode 300 is shown in contact with the scalp of the subject user 110. This may be at a localized region of the head of the subject user 110, for example, the occipital region at the rear of the skull. For comfort and continuous contact, the active dry electrode 300 may also be provided with a coil spring arrangement 306.

The amplification at each electronic circuit 304 makes it possible to adapt the amplitude of the signals to the characteristics of the ADC circuit (104 in FIGS. 1 & 2), and to obtain a maximum resolution during the conversion.

In some examples, where a more compact EEG device is used, active dry electrodes, such as one or more of active dry electrode 300 in FIG. 3, are attached to or embedded in an elasticated head band or helmet worn about the head, thereby biasing the sensors against the head. As many users will attest, the band or helmet itself may give rise to discomfort and may be inconvenient in regular use-interfering with other wearable devices, headgear and ornamentation the wearer might already carry or wear (such as hats, jewelry, hairstyles, headphones, face masks, etc.)

It is a further challenge for any headset arrangement to be able to apply a predetermined biasing force to the sensors regardless of the head shape and/or head dimensions of the wearer.

In some examples, the electrodes of an EEG device are mounted on an eyewear device.

Placing the electrodes of an EEG device in an eyewear device form factor does mean that the device has a more acceptable appearance (which removes a barrier to adoption) while also facilitating secure wearability. If the eyewear device is configured to receive and process the output of the EEG electrodes to infer the wearer's intention though attention to a given visual stimulus, the mounted EEG device becomes an input means for the eyewear device.

Eyewear devices, such as smart glasses, Augmented Reality (AR) glasses, Virtual Reality (VR) glasses and the like, accept input via a separate input device such as a smartphone or through voice activation and output visual stimuli through their embedded displays. The synergy of EEG device with such eyewear devices creates an autonomous system for visual BCI (i.e., using EEG signals to measure the neural response and the AR/VR display system to stimulate the user's visual cortex).

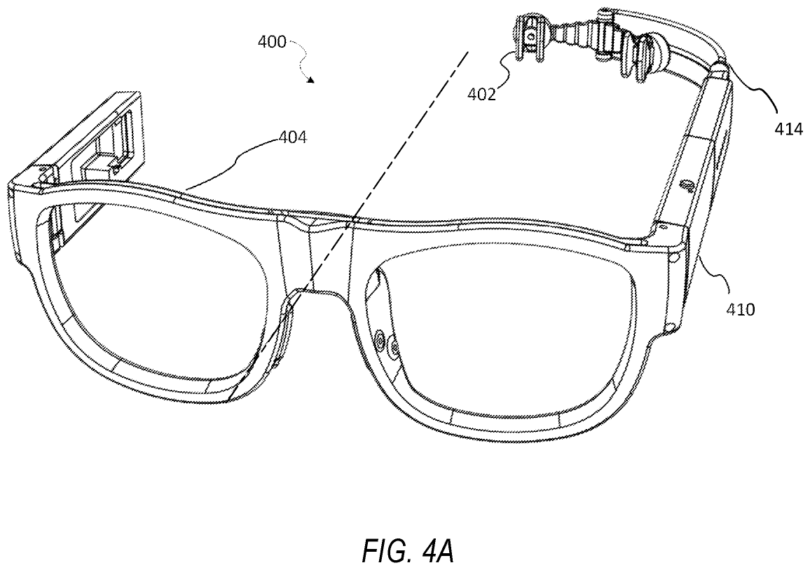

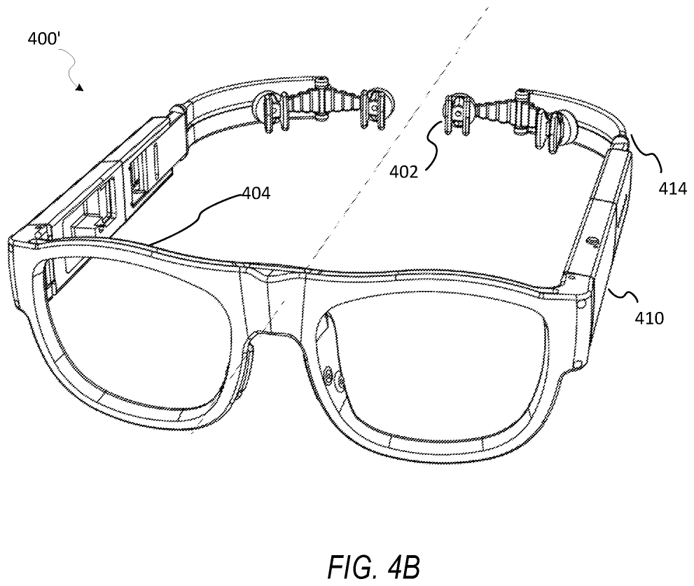

FIGS. 4A and 4B show eyewear devices 400, 400′ according to an embodiment of the present disclosure. The eyewear devices are each formed of a frame 404 and at least one temple arm 410. While FIG. 4A illustrates only one temple arm 410 joined to the frame 404, the eyewear device 400′ of FIG. 4B is provided with a second temple arm at the opposite side of the frame 404 so that the temple arms pass over or across respective ears of a wearer, when worn.

In this disclosure, directional terms such as front, back, forward(s), and rearward(s) are to be understood with reference to a direction of view of a user when the eyewear device is worn. Likewise, the terms horizontal and vertical with reference to different features of the eyewear device are to be understood as corresponding to the orientation of the eyewear device when it is worn on the face of a wearer looking forwards.

Biopotential measurement components of a biopotential measurement system 402 (e.g., EEG sensors similar to the active dry electrode 300 in FIG. 3) are mounted on a rearward extension 414 of one or more temple arms of the at least one temple arm 410 of the eyewear device 400 (FIG. 4A shows only one temple arm with the biopotential measurement system 402 while FIG. 4B shows mounted components on each of two temple arms). As a result, the biopotential measurement system 402 is located close to the occipito-temporal (i.e., visual) cortex of the wearer's brain. Furthermore, by mounting the components to a substantially rigid eyewear device, the features of the eyewear device that locate optical components (e.g., displays, lenses, and the like) of the eyewear device over the wearer's eyes also ensure that the components are naturally placed over the relevant region of the wearer's brain.

The biopotential measurement components of the biopotential measurement system 402 form an electrode arrangement suitable for measuring biopotential signals arising from visual activity in the wearer's brain. These measured signals may be processed further in a brain-computer interface (BCI) system, so that the measured biopotential signals may be interpreted as control input into a processing environment such as a user interface of a computer system and/or a wearable device.

By locating biopotential measurement components on the rearward extension 414, they become less obtrusive, even invisible, especially when the electrodes are hidden by hair. Once worn, the eyewear device 400 may appear indistinguishable from a pair of glasses or smart glasses.

Indeed, the eyewear device of the present disclosure may be integrated in augmented reality AR glasses facilitating brain computer interface (BCI) interactions on display elements of the AR glasses. In some examples, the integration of the eyewear device in smart glasses may be used to facilitate control over IoT objects, as well as, or instead of, the virtual objects displayed in the AR display.

Furthermore, the eyewear device of the present disclosure takes advantage of the fact that biopotential signals can be detected at the side of the wearer's head at sufficient strength for use in a BCI system. As a result, the temple arm extension 414 of the eyewear device need not extend all the way to the midline (illustrated as a dashed line extending through the bridge of the eyewear device 400, 400′) at the back of the head in order to access usable biopotential signals.

The eyewear device of the present disclosure is thus suitable for a wider range of users, including people who need to have their head supported by a headrest, which would rule out the use of a biopotential measurement device that had to be located at the back of the head at or near the midline. Wheelchair users, for example, may benefit from a visual BCI system to control their chair but would find that compact EEG devices worn at the back of their heads would prevent them from having adequate head support. More widely, comfort is also improved for users in vehicle seats where a heat rest is a requirement for safety. The absence of a cage or mesh of electronic components and/or battery packs at the back of the head leads to greater comfort for many wearers.

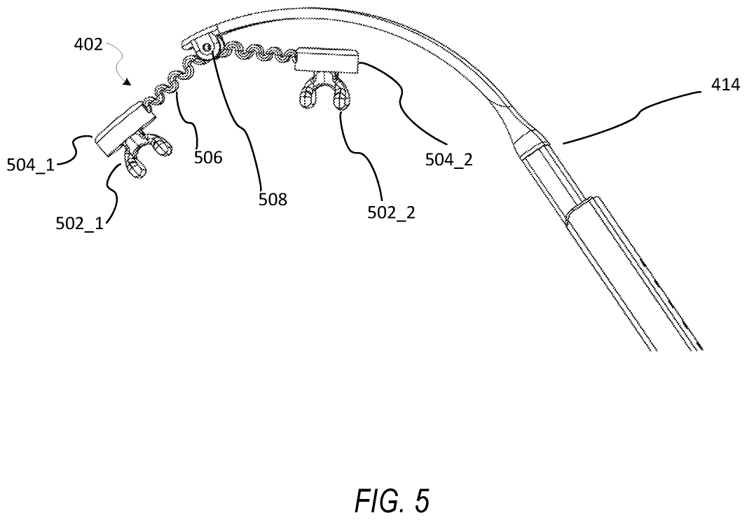

FIG. 5 shows detail of an electrode arrangement for an eyewear device according to the present disclosure, such as an electrode arrangement of a biopotential measurement system 402 of FIGS. 4A and 4B. The electrode arrangement of the biopotential measurement system 402 is mounted at a rearward extension 414 of a temple arm of the eyewear device.

In the illustrated example, an electrode arrangement of the biopotential measurement system 402 is an arrangement of active dry electrodes such as the electrode illustrated in FIG. 3. The biopotential measurement components of the biopotential measurement system 402 comprise a plurality of sensors 502_1, 502_2 each with a respective electronic circuit 504_1, 504_2 (analogous to the electronic circuit 304 in FIG. 3). The electronic circuits 504 each implement the “active” part of the active dry electrode for the corresponding sensor 502 electronically filtering and amplifying the biopotential signal measured at the sensor 502. In some examples, a mirror image biopotential measurement system may be provided on a rearward extension to the other temple arm: resulting in a pair of biopotential measurement electrodes corresponding to sensor 502 on each temple arm, or four biopotential measurement electrodes in total. In some examples, fewer or more such electrodes may be provided on each temple arm extension. Furthermore, it is not essential that the same number of electrodes be provided on each temple arm extension. Asymmetric arrangements are also contemplated.

In some examples, such as but not limited to the examples illustrated in FIGS. 4A, 4B and 5, the electronic circuits 504 are implemented in small-form factor printed circuit board (PCB) components adjacent to the sensors 502 they serve. The PCBs are conveniently housed in a protective case.

The biopotential measurement components of the biopotential measurement system 402 further comprise a resiliently deformable support 506 to which the sensors 502 and electronic circuits 504 are fixed. The sensors themselves may be formed of a conductive polymer, for example. The sensors may take a number of shapes including the blade-like shape described in co-pending international patent application, WO 2021/099382 A1, the contents of which are incorporated herein by reference.

Physical and electrical connection is established between the support 506 (and thence to the electronic circuits 504) and the temple arm extension 414 at a mounting point 508. In FIG. 5, the mounting point 508 is shown as a hinge, however flexibility in the support and/or the temple arm extension mean that rotation about a vertical axis at this mounting point this is not essential.

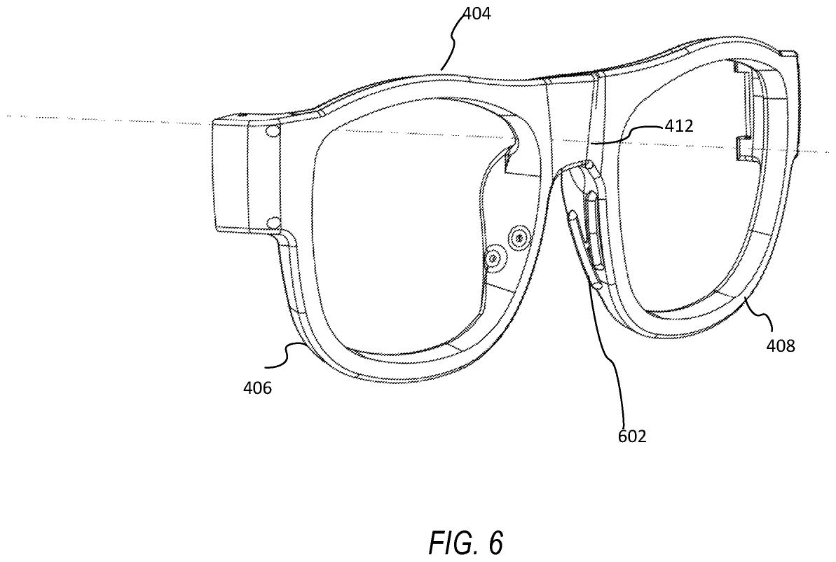

FIG. 6 shows detail of the frame of an eyewear device according to the present disclosure, such as the eyewear device 400 of FIG. 4A or eyewear device 400 of FIG. 4B.

The eyewear frame 404 includes left and right optical element holders 406, 408 (suitable for holding optical elements, not shown, such as corrective and/or protective lenses or display elements). The optical element holders 406, 408 are joined at a bridge 412. When worn, the optical elements are positioned so that they are substantially coplanar in front of the wearer's eyes (in a plane perpendicular to the wearer's line of sight when looking forward) with the bridge 412 positioned above or in front of the nose of the wearer. To align the frame on the wearer's face, the frame includes nose pads 602 configured to sit at either side of the nose, typically against the nasal bone.

In some examples of the eyewear device of the present disclosure, a reference electrode (also an active dry electrode) is located on one of the nose pads 602 of the eyewear device. In some examples, a bias electrode (not shown in FIG. 6) may be provided on the other nose pad.

The nose can be a good place to locate a reference electrode as it is comparatively distant from the biopotential measurement electrodes at the distal end of the temple arm extension and because the skin over the nose is substantially hair-free, ensuring good contact. The reference electrode may serve the same purpose as reference electrode 208 in the scheme illustrated in FIG. 2.

A nose pad location for the reference electrode is however exposed to a number of possible additional sources of measurement artefacts. The nose is, after all, close to other facial features so that wearers with particularly mobile faces, experiencing facial tics and/or eye blinks may find that even those small movements add significant noise to the measured signal. In some examples, the reference electrode is placed, instead, just above one ear within the inner portion of one of the temple arms (not illustrated). The bias electrode may conveniently be placed just above the other ear on the inner portion of the other one of the temple arms.

Whichever location is adopted for the reference electrode, the placement of a reference electrode at a distance from the biopotential measurement electrodes in the biopotential measurement components of the biopotential measurement system 402 improves the signal to noise ratio (SNR) compared to EEG devices that are confined for aesthetic and/or practical reasons to a single area of the head.

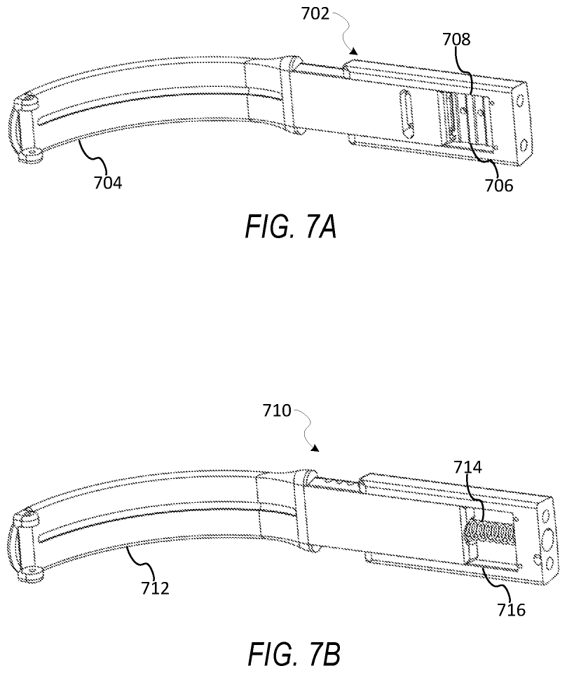

FIG. 7A illustrates a temple arm adjustment arrangement for an eyewear device in accordance with some examples. In some examples, a maximum angle between a frame and a temple arm 702 of an eyewear device can be adjusted. In some examples, a dimension of a bridge of the frame and a size and shape of an optical element holder of the eyewear device can be adjusted.

In some examples, a temple arm extension 704 for mounting a biopotential measurement system having biopotential measurement components is held against a rear portion of the head of a user with a reliable and secure, but comfortable, biasing force. In some examples, an additional facility for adjustment of a length of the temple arm 702 is provided. In some examples, this is done by adjusting a length of the temple arm 702 to predefined fixing positions 706 that adjust a depth of engagement of the temple arm extension 704 in a longitudinal slot 708 of the temple arm 702.

FIG. 7B illustrates a temple arm adjustment arrangement for an eyewear device in accordance with some examples. A temple arm 710 includes a temple arm extension 712 for mounting a biopotential measurement system having biopotential measurement components. The temple arm extension 712 is held against a rear portion of the head of a user with a biasing force. The biasing force is provided automatically by means of a spring 714 urging a length of the temple arm to a minimal position by urging a portion of the temple arm extension 712 into a longitudinal slot 716 of the temple arm 710.

Although described through a number of detailed examples, the portable devices for the acquisition of electroencephalographic signals according to the present disclosure comprise various variants, modifications and improvements which will be obvious to those skilled in the art, it being understood that these various variants, modifications and improvements fall within the scope of the subject of the present disclosure, as defined by the following claims.

1. An electroencephalogram (EEG) device comprising:
a frame;
a first temple arm connected to a first side of the frame, and a second temple arm connected to a second side of the frame opposite the first side of the frame;
a reference electrode placed above one ear of a user within an inner portion of the first temple arm; and
a bias electrode placed above another ear of the user within an inner portion of the second temple arm; and
a biopotential measurement system comprising one or more electrodes, the biopotential measurement system mounted on a rearward extension of at least one of the first temple arm and the second temple arm, the biopotential measurement system positioned proximate to an occipito-temporal cortex of a wearer's brain, the reference electrode and the bias electrode each separated from the one or more electrodes of the biopotential measurement system.

2. The EEG device of claim 1, wherein the frame comprises:
a first optical element holder and a second optical element holder joined by a bridge.

3. The EEG device of claim 2, wherein a first nose pad is configured to sit at a first side of a nasal bone of a user of the EEG device.

4-5. (canceled)

6. The EEG device of claim 1, wherein the one or more electrodes are configured to be in contact with a scalp of a user of the EEG device.

7. The EEG device of claim 6, further comprising:
an acquisition subsystem operably connected to the reference electrode of the frame and the one or more electrodes of the biopotential measurement system, wherein the acquisition subsystem is configured to capture and digitize EEG signals in the EEG device.

8. The EEG device of claim 7, wherein the acquisition subsystem is further configured to use the reference electrode as a reference for the one or more electrodes of the biopotential measurement system.

9. The EEG device of claim 8, wherein the acquisition subsystem is further configured to take an average of activity of the reference electrode and the one or more electrodes of the biopotential measurement system as a reference.

10. The EEG device of claim 6, wherein the reference electrode and the one or more electrodes of the biopotential measurement system are active dry electrodes.

11. The EEG device of claim 1, wherein the temple arm comprises a plurality of predefined fixing positions for adjusting a depth of engagement of the rearward extension in a longitudinal slot of the temple arm.

12. The EEG device of claim 1, wherein the temple arm comprises a spring urging a portion of the rearward extension into a longitudinal slot of the temple arm.

13. The EEG device of claim 1, wherein the EEG device comprises AR glasses.

14. An electroencephalogram (EEG) device comprising:
a frame;
a first temple arm connected to a first side of the frame, the first temple arm comprising a reference electrode-placed above one ear of a user and within an inner portion of the first temple arm;
a second temple arm connected to a second side of the frame, the second side of the frame opposite the first side of the frame, the second temple arm comprising a bias electrode placed above another ear of the user and within an inner portion of the second arm;
a first biopotential measurement system comprising a first one or more electrodes, the first biopotential measurement system mounted on a first rearward extension of the first temple arm, the first biopotential measurement system positioned proximate to a first occipito-temporal cortex of a wearer's brain, the reference electrode separated from the first one or more electrodes of the first biopotential measurement system; and
a second biopotential measurement system comprising a second one or more electrodes, the second biopotential measurement system mounted on a second rearward extension of the second temple arm, the second biopotential measurement system positioned proximate to a second occipito-temporal cortex of the wearer's brain, the bias electrode separated from the second one or more electrodes of the second biopotential measurement system.

15. The EEG device of claim 14, wherein the frame comprises:
a first optical element holder and a second optical element holder joined by a bridge.

16. The EEG device of claim 15, wherein the frame further comprises:
a first nose pad configured to sit at a first side of a nasal bone of a user of the EEG device.

17-18. (canceled)

19. The EEG device of claim 15, wherein the first biopotential measurement system comprises:
first one or more electrodes in contact with a scalp of a user of the EEG device.

20. The EEG device of claim 19, wherein the second biopotential measurement system comprises:
second one or more electrodes in contact with the scalp of the user of the EEG device.

21. The EEG device of claim 1, wherein the EEG device comprises AR glasses.

22. The EEG device of claim 1, wherein the biopotential measurement system further comprises:
a one or more electronic circuits operably connected to the one or more electrodes; and
a resiliently deformable support to which the one or more electrodes and the one or more electronic circuits are fixed.

23. The EEG device of claim 14, further comprising:
a first one or more electronic circuits operably connected to the first one or more electrodes;
a first resiliently deformable support to which the first one or more electrodes and the first one or more electronic circuits are fixed;
second one or more electronic circuits operably connected to the second one or more electrodes; and
a second resiliently deformable support to which the second one or more electrodes and the second one or more electronic circuits are fixed.
